(19) United States
(12) Patent Application Publication
(10) Pub. No.: US 2012/0167895 A1
(43) Pub. Date: Jul. 5, 2012

(54) THERAPEUTIC SLEEP DEVICE FOR DISCOURAGING SLEEPING IN A SUPINE POSITION

(76) Inventors: Sanjiv Tewari, Akron, OH (US); John Verdile, Medina, OH (US); Hans Dorfi, Akron, OH (US)

(21) Appl. No.: 12/983,453

(22) Filed: Jan. 3, 2011

(51) Int. Cl. A61F 5/56

(52) U.S. Cl. 128/845

(57) ABSTRACT

A therapeutic sleep device worn about an individual's torso and intended to discourage an individual from sleeping in a supine position includes a body securing member and a sleep fin. The body securing member has an interior side and a posterior side. The sleep fin includes a supine-suppression fin providing a right convex surface and a left convex surface tapering from a wider base region to a narrower peak region at which the right and left convex surfaces join at an apex. The apex and at least a portion of the right and left convex surfaces extend beyond said posterior side of the body securing member. A spine indentation formed in the sleep device can help to increase the comfort and improve the securing of the device to a individual.

### FIELD OF THE INVENTION

The present invention generally relates to a therapeutic sleep device, worn by an individual to discourage the individual's desire and ability to sleep in the supine position. The device is intended to be worn about an individual's torso in such a manner that it is affixed to the individual's back through the use of a strap, belt, vest or harness.

### BACKGROUND OF THE INVENTION

According to data from the National Sleep Foundation (District of Columbia, USA), snoring and sleep apnea affect up to a quarter of the population in the United States. Snoring occurs when increased resistance in the nasal passages or upper airway causes those structures to vibrate. If the resistance is particularly high, the upper airway collapses upon itself leading to a cessation of breathing or “apnea.”

Both snoring and sleep apnea have been associated with serious medical consequences. Sleep apnea, in particular, can lead to hypertension, strokes, heart attacks, heart arrhythmias, congestive heart failure, hormonal imbalances, mood disorders, and even sudden death at night. Snoring has also been linked to adverse cardiovascular and neurocognitive sequelae. Treatment options for sleep apnea include continuous positive airway pressure (CPAP) delivered via a tight fitting mask. In some extreme cases, sleep apnea may even be treated with upper airway surgery. Because CPAP must be delivered by a mask worn by the apnea sufferer, this treatment process can be ineffective (or at least less effective) when the sufferer fails to consistently wear the mask and/or fails to properly fit the mask prior to sleep. Surgery, in addition to being a drastic measure, often is not successful in eliminating occurrences of sleep apnea.

Snoring and sleep apnea both typically worsen when one lies in the supine position, i.e., on one's back, while sleeping. In this position, there is a greater propensity for the upper airway structures to collapse posteriorly under the influence of gravity, and upper airway resistance is consequently increased in the supine position. Some patients that suffer from sleep apnea only have apnea while on their backs. Thus, for many individuals, snoring and/or sleep apnea can be reduced or even prevented by taking measures to avoid a supine sleeping position.

The prior art provides limited options for preventing people from sleeping in the supine position. In sleep laboratories where snoring and sleep apnea are studied and treated, patients are sometimes advised to place a tennis ball in a sock and sew it to the back of their night clothing. The tennis ball is worn in this manner in hopes that the pressure of the tennis ball against one's back will urge one to roll over to a non-supine sleeping position. Needless to say, this method is fairly crude. Because the tennis ball is sewn to clothing, it can move around quite easily, just as one's clothing moves relative to their body as they roll around or change position in bed. Additionally, the tennis ball itself is fairly soft, and loses pressure over time, such that it is quite possible for one to sleep in the supine position directly on top of the tennis ball without being forced out of that position by the discomfort of the tennis ball. Also the localized pressure of a ball-shaped object can potentially lead to injury to the back or spine.

U.S. Pat. No. 4,958,644 teaches a device that is strapped or otherwise attached to an individual's back to discourage supine sleeping. Blunted prods are recessed in a foam material such that, when the individual wearing the device rolls over onto his back, the foam compresses and the prods stick the user of the device in the back. The discomfort felt by being stuck with the prods is intended to cause the wearer to avoid the supine position. This approach is quite invasive and therefore undesirable.

U.S. Pat. No. 6,289,893 discloses a harness-type snore reducer jacket wherein an elongate cushion of low density foam is bookended by compressible columns of high density foam such that the high density foam is placed proximate the sides of the individual's back. These columns of high density foam are intended to prevent the wearer from rolling into the supine position. By placing the columns near the wearer's sides, this device provides a structure that would actually support the wearer in a supine position, with the side columns cradling the torso.

Similarly, U.S. Pat. No. 6,926,008 discloses a snore relief belt that includes stop blocks that are to be worn near the lateral sides of the torso to frustrate rolling over to a supine position during sleep. This structure is functionally identical to the prior art shown in U.S. Pat. No. 6,289,893 and also would support the wearer in a supine position, which is the opposite of the intended purpose of the present invention.

U.S. Pat. No. 6,357,444 discloses a device mounted by straps onto an individual's back to limit the ability of the individual to move when lying down. The pad is taught as being rectangular or trapezoidal in shape. This device generally provides a protrusion at an individual's back to frustrate sleeping in the supine position. This approach suffers from some drawbacks. First, the structure and shape of the device prevent or at least significantly frustrate one's ability to roll over, which is a natural tendency when one sleeps. When rolling toward the supine position, the shape of the device is such that the pad abruptly engages the support surface (e.g. mattress), with a significant area of contact between a flat pad surface and a flat support surface, and the individual thus experiences a transition from no resistance of movement (when the pad does not touch the support surface) to full resistance of movement (when the pad and support surface come into contact). This resistance will at least significantly frustrate movement toward or through the supine position, thus frustrating an individual's natural tendencies to roll over during sleep. Second, there is no mechanism for protecting the spine from pressure that would be transmitted if the wearer were in the supine position, even transiently. Third, there is the possibility that obese individuals, who are more prone to developing sleep apnea and snoring, will be able to sleep in the supine position especially if they use a softer mattress. Under an individual's weight (particularly if obese), the flat posterior surface of the device will tend to sink into the mattress and the flat surface would serve as a stable platform thus permitting the individual to remain in the supine position, thus obviating any beneficial effect.

U.S. Pat. No. 7,134,435 and U.S. Published Application 2006/0201518 are related. Each of these references discloses a belt-type sleeping apparatus, and the patent focuses on the belt itself, while the published application focuses on a method of using the belt. The belt has a rectangular cushion section wherein the mid-section is softer than the end segments. The mid section is taught as also housing certain “controls” such as a massage pad or nerve stimulation device. With this device it would not be difficult for a wearer to lie in a supine position, especially because the lower back easily flexes to accommodate the relatively small cushion section. Again, this device prevents rolling, lacks spinal protection, and could be a stable platform for sleeping supine if it were compressed into a mattress by an obese individual.

U.S. Published Application 2007/0256695 discloses a belt having elastic portions provided so that the belt can fit various waist sizes. It includes a specifically shaped “position control member” with sidewalls that angle outwardly such that the position control member gets larger as it extends away from an individual's back. This device also would not permit rolling over and it offers no spinal protection. The latter is particularly important because of the broad posterior base which transmits force to a narrower anterior base abutting the spine. The size of the small posterior protrusion is insufficient to prevent an obese patient from compressing it into a mattress and creating a stable, relatively flat platform for sleeping.

### SUMMARY OF THE INVENTION

In one embodiment, this invention provides a therapeutic sleep device worn about an individual's torso and intended to discourage an individual from sleeping in a supine position. The therapeutic sleep device includes a body securing member and a sleep fin. The body securing member has an interior side and a posterior side. The sleep fin includes a supine-suppression fin providing a right convex surface and a left convex surface tapering from a wider base region to a narrower peak region at which the right and left convex surfaces join at an apex. The apex and at least a portion of the right and left convex surfaces extend beyond said posterior side of the body securing member.

In another embodiment, this invention provides therapeutic sleep device worn about an individual's torso and intended to discourage an individual from sleeping in a supine position. The therapeutic sleep device includes a body securing member and a sleep fin. The body securing member has an interior side and a posterior side. The sleep fin includes a torso base portion defining a spine indentation centered between a right torso wing and a left torso wing, and the spine indentation is aligned with the apex.

In yet another embodiment, this invention provides therapeutic sleep device worn about an individual's torso and intended to discourage an individual from sleeping in a supine position. The therapeutic sleep device includes a body securing member and a sleep fin. The body securing member has an interior side and a posterior side. The sleep fin includes a supine-suppression fin providing a right convex surface and a left convex surface tapering from a wider base region to a narrower peak region at which the right and left convex surfaces join at an apex, and a torso base portion defining a spine indentation centered between a right torso wing and a left torso wing, the spine indentation being aligned with the apex. The apex and at least a portion of the right and left convex surfaces extend beyond the posterior side of said body securing member.

In particular embodiments, the shape of the fin is designed to gradually engage the sleep support surface as the person attempts to roll into a supine position. Due to the narrow apex the supine sleep position is unstable and a person using the device will continue to roll into a position sufficiently angled from the supine position to be effective. The soft cushion of the backing material ensures the comfortable use of the device as a person rolls through a supine position because of its ergonomic design, which reduces back pressure.

The body securing member is to be understood to be any appropriate belt, harness, vest, shirt or other strap system that can secure the therapeutic sleep device to an individual's body. With respect to the body securing member, as used herein, the “interior side” is to be understood as referring to the portion of the body securing member that faces inwardly, i.e., toward the user's torso, and the term “posterior side” is to be understood as the portion of the body securing member that faces outwardly.

The supine-suppression fin is so named because it has a general fin shape and because it is intended to discourage and suppress one's desire and ability to sleep in the supine position. The torso base portion and the torso wings are so named because, in those instances where the therapeutic sleep device is worn high on the torso, those “interscapular” elements are intended to lie between the individual's scapulae, preferably contacting the scapulae, such that the device is anchored in that position.

In some embodiments, the soft interior back-contacting region of the therapeutic sleep device is provided by the body securing member, at the interior side thereof. In other embodiments, the soft interior back-contacting region is provided by the torso base portion.

In some embodiments, the torso base portion is formed as a unitary piece with the supine-suppression fin. Whether they are of a unitary piece or are separate elements joined together, the torso base portion and the supine-supression fin together form what is termed herein a “sleep fin.”

In some embodiments, the torso base portion is secured to the posterior side of the body securing member, and is removable therefrom. In particular embodiments, the base portion is secured to the body securing member by hook-and-loop type fasteners. In yet other embodiments, the body securing member includes an aperture, and an integral torso base portion and supine-suppression fin is positioned with the torso base portion at the interior side of the body securing member and the supine-suppression fin extends through the aperture to be present at the posterior side of the body securing member.

### BRIEF DESCRIPTION OF THE DRAWINGS

FIG. 1 is a perspective view of a first embodiment of the therapeutic sleep device;

FIG. 2 is a front elevational view of the posterior side of the body securing member of the device;

FIG. 3 is a front elevational view of an interior side of the body securing member;

FIG. 4 is a perspective view of the sleep fin portion of the first embodiment;

FIG. 5 is a top plan view of the sleep fin portion of the first embodiment;

FIG. 6 is a rear elevational view of the sleep fin portion of the first embodiment;

FIG. 7 is a cross section showing how the first embodiment of the therapeutic sleep device would be worn by an individual;

FIG. 8 is a cross section, as in FIG. 7, but showing a sleep position in which the wearer is supported off of the supine position by the therapeutic sleep device;

FIG. 9 is a perspective view of a second embodiment of a therapeutic sleep device in accordance with this invention;

FIG. 10 is a top plan view of a second embodiment of a sleep fin having an integral torso base portion and supine-suppression fin and employed in the second embodiment of the therapeutic sleep device of FIG. 9;

FIG. 11 is a cross section showing how the second embodiment of the therapeutic sleep device would be worn by an individual; and

FIG. 12 is a top plan view of a third embodiment of a sleep fin and how it would secure to a body securing member such as that shown with respect to the first embodiment of the therapeutic sleep device.

### DETAILED DESCRIPTION OF ILLUSTRATIVE EMBODIMENTS

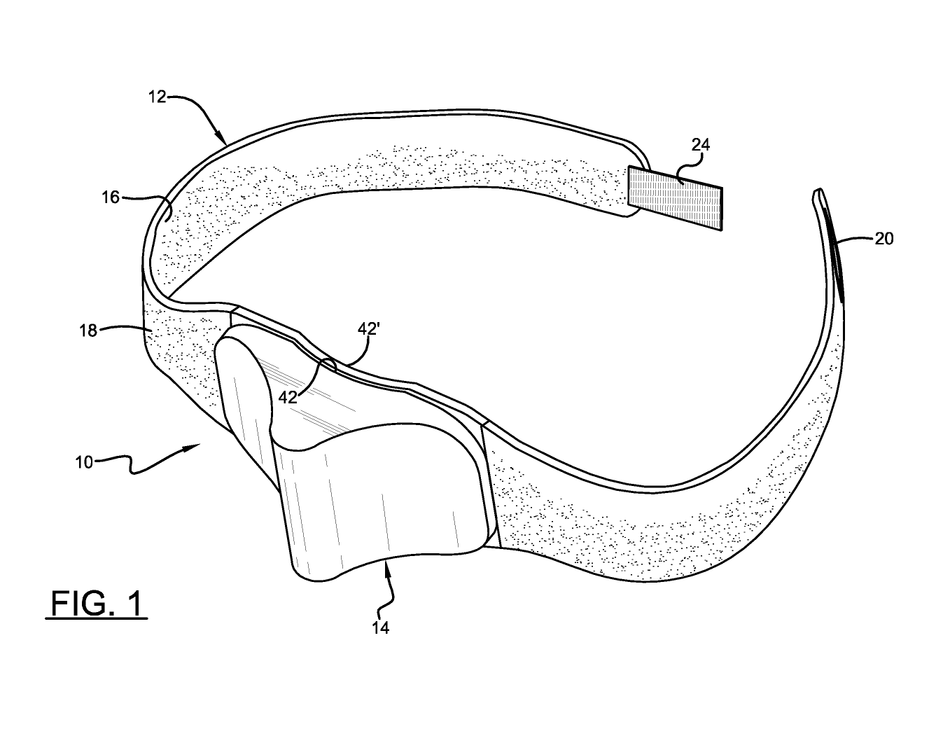

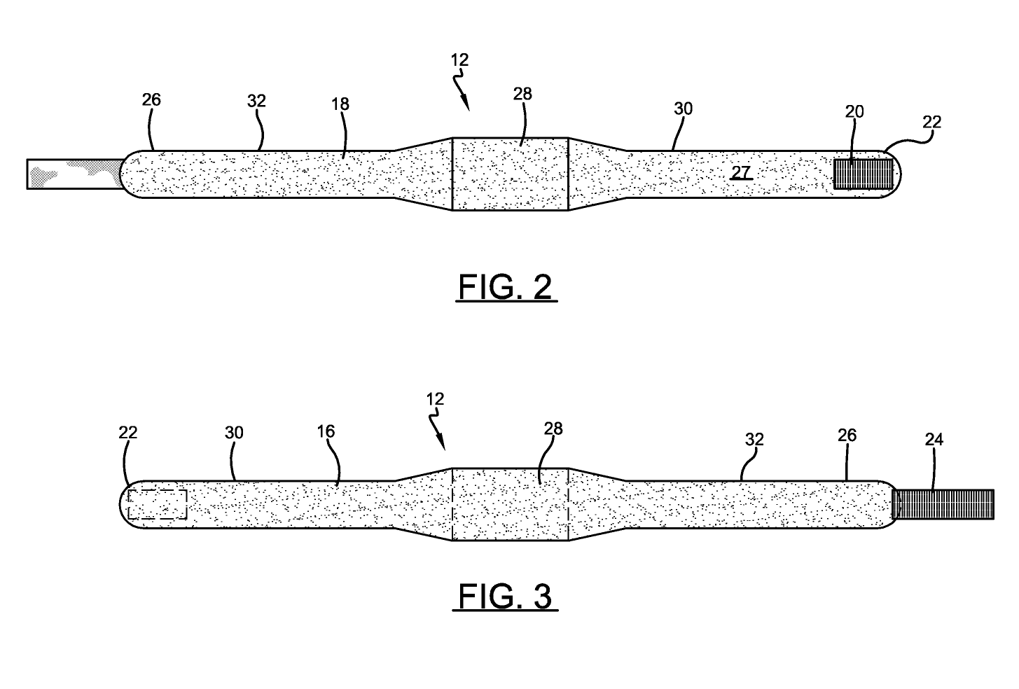

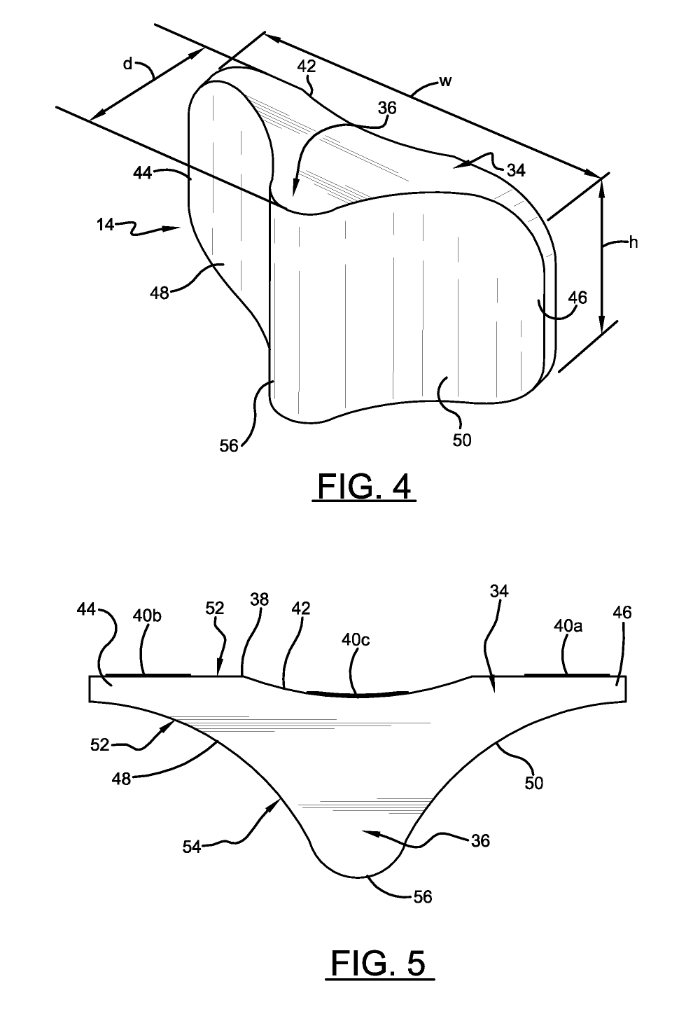

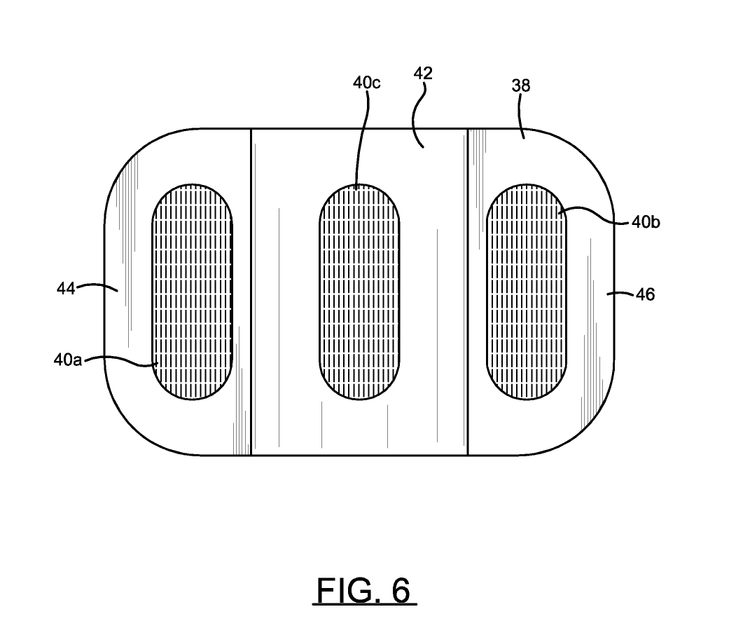

With reference FIGS. 1-6, a first embodiment of a therapeutic sleep device is shown and designated by the numeral 10. The therapeutic sleep device 10 includes a body securing member 12 and a sleep fin 14. In this embodiment, the body securing member 12 is generally a belt, but it will be appreciated as already disclosed above that the body securing member may be any appropriate belt, harness, vest, shirt or other strap system that can secure the therapeutic sleep device to an individual's body. As seen in FIGS. 2 and 3, the body securing member 12 provides an interior side 16 and a posterior side 18. The interior side 16 is the side of the body securing member that is placed against the wearer's torso, and the posterior 18 is the opposite side that faces away from the wearer's torso. The body securing member 12 is preferably sized in accordance with the individual that is to wear the therapeutic sleep device 10, and, to that end, can be of various sizes as generally appreciated in the arts of belts, harnesses, vests, shirts etc.

For belt-type securing members, any suitable fastening means could be employed in the art of belts to secure the belt to an individual's torso. Common belt buckles might be employed or the belt might even be tied in a knot against the individual's torso to secure it. Harness-type securing members would be similar to belt-type securing members in that straps would be employed to encircle portions of the wearer's body and firmly secure the sleep fin 14 at the wearer's back. Vests, shirts and other clothing type securing members would be appropriately sized to secure the sleep fin 14 at the wearer's back. The placement of the sleep fin 14 relative to the wearer's torso and spine will be taught more fully herein.

In this particular embodiment, hook members 20 are provided proximate the distal first end 22 of the body securing member 12, at the posterior side 18 thereof. Hook members 24 are also provided proximate the distal second end 26 of the body securing member, though, in this particular embodiment, they are provided on a patch of fabric that actually extends from the distal second end 26. The hook members 24, being considered a part of the body securing member 12, are provided at the interior side 16 of the body securing member 12, such that the hook members 20 face in a different direction than do hook members 24. It should be appreciated that the hook members 24 could be provided directly on the second distal end 26, just like the hook members 20 are placed directly on the first distal end 22. It should be understood that these “hook members” are hook portions of a hook-and-loop type fastener (e.g., Velcro™). In this particular embodiment, the exterior surface of the body securing member 12 is preferably made of a material that can act as a loop portion of a hook-and-loop type fastener, such that the hook members 20 and 24 secure to the loop fabric 27 of the body securing member 12 when the body securing member 12 is properly wrapped about a wearer's torso, as will be explained below.

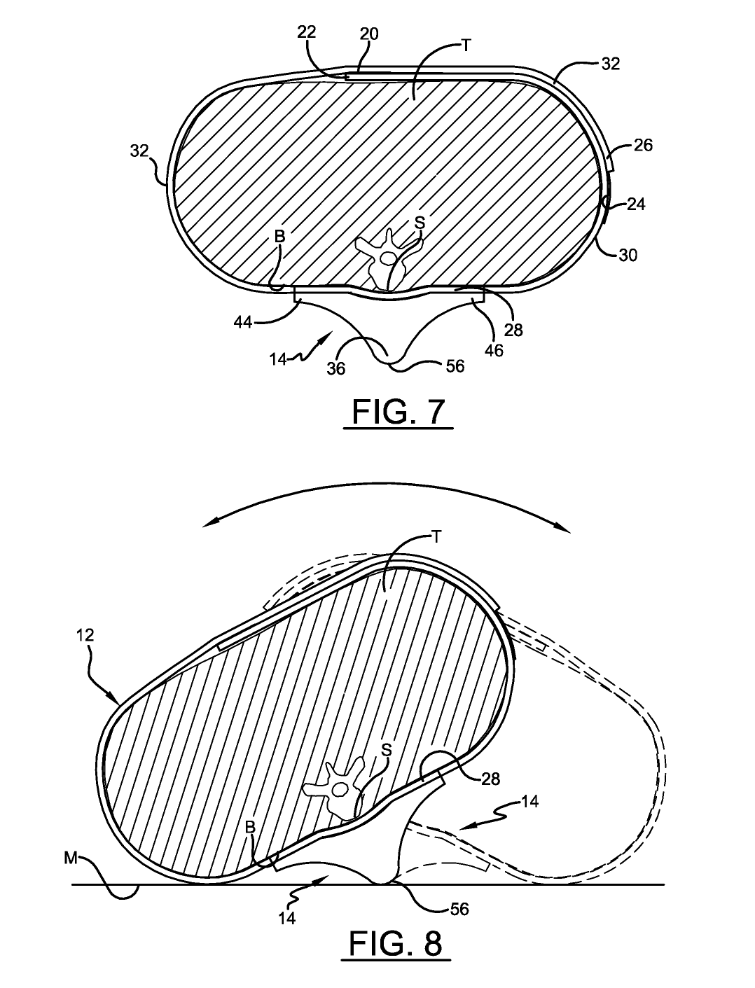

In this embodiment, the body securing member 12 includes a back pad portion 28 from which extends a first extension 30 and a second extension 32, with the first extension 30 providing the distal first end 22 and hook members 20 and the second extension 32 providing the distal second end 26 and hook members 24. With reference now to FIG. 7, it can be seen that this embodiment is wrapped around a wearer's torso T by placing the back pad portion 28 against the individual's back B, with the back pad portion 28 preferably centered thereon about the spine S. The first extension 30 is then wrapped around the wearer's chest in a snug manner, after which the second extension 32 is wrapped over the first extension 30 in a snug manner. In this way, the hook members 20 at the posterior side 18 of body securing member 12 are brought into contact with the loop fabric 27 of the second extension 32 to secure thereagainst, and, similarly, the hook members 24 at the anterior side 16 of the body securing member 12 are brought into contact with the loop fabric 27 of the first extension 30 to secure thereagainst. Thus, this embodiment of the body securing member 12 can be secured to the wearer's torso T. Additionally, this securing member 12 can fit people of various sizes because the first extension 30 and second extension 32 can simple overlap to a greater or lesser degree according to the size of the individual wearing the device 10. This provides a more universal sizing capability (i.e., a one-size-fits-all option).

In this embodiment, the sleep fin 14 is secured to the body securing member 12 at the back pad portion 28 thereof. With reference to FIGS. 4-6, the sleep fin 14 includes a torso base portion 34 and a supine-suppression fin 36, and the interior base surface 38 of the torso base portion 34 includes multiple patches of hook members 40a, 40b and 40c, which can secure the sleep fin 14 to the back pad portion 28 because the body securing member 12 of this embodiment has an exterior surface made of a loop fabric 27. It should be appreciated that, as used herein, a loop fabric is any fabric that is suitable for interacting with hook members to act as a hook-and-loop fastener. It should also be appreciated that, in accordance with other embodiments, the sleep fin 14 could be otherwise secured to the back pad portion 28 of a body securing member. Indeed, as already mentioned, various means for securing the body securing member to a torso may be employed, and, thus, various materials could be employed to form the body securing member 12.

The torso base portion 34 is shaped to define a spine indentation 42 at the interior base surface 38. The spine indentation 42 is centered between a left torso wing 44 and a right torso wing 46. In this particular embodiment, the patch of hook members 40a is provided on the interior base surface 38 at left torso wing 44, and the patch of hook members 40b is provided on the interior base surface 38 at the right torso wing 46. The patch of hook members 40c is provided within the spine indentation 42. By providing the patches of hook members 40a, b and c in this manner, the back pad portion 28 can be made to conform to the shape of the interior base surface 38 of the torso base portion 34 such that the back pad portion 28 defines a spine indentation 42′ (FIG. 1), when the sleep fin 14 is fastened to the back pad portion 28.

In some embodiments, the back pad portion 28 is made of any soft material that will cushion the pressure of the sleep fin 14 against the wearer's back. In other embodiments, the material will also readily conform to the shape of the spine indentation 42 on the interior base surface 38 when the torso base portion 34 is secured to the back pad portion 28 if formed of high density foam. In other embodiments, the foam has a force deflection of from 25 to 75 pounds (lbs) as measured by ASTM D3574. In accordance with other embodiments the foam has a force deflection of from 30 to 65 lbs (ASTM D3574), and, in yet other embodiments from 40 to 60 lbs (ASTM D3574).

The supine-suppression fin 36 provides a left convex surface 48 and a right convex surface 50, both of which taper from a wider base region 52 to a narrower peak region 54. At this peak region, the left and right convex surfaces 48, 50 join at an apex 56, which is preferably slightly rounded, as shown, though it could be a sharper point, if desired. The apex 56 is aligned with the spine indentation 42. As seen in FIG. 7, the spine indentation 42 provides room for a wearer's spine S, and thereby increases the comfort of the therapeutic sleep device 10, when worn, and helps to ensure that the therapeutic sleep device 10 remains in place, with the apex 56 aligned with the wearer's spine.

The width (w, FIG. 4) of the torso base portion 34 is sized so that it provides position stability in the lateral direction of the wearer's back. The width itself limits lateral sliding but additional stability is achieved when worn in the preferred position across the upper torso, where it engages with the wearer's scapulae. In particular embodiment, the torso base portion 34 is sized specifically to extend between a given individual's scapulae. That is, the distance between scapulae is measured and the torso base portion 34 is laterally sized accordingly. In other embodiments, it has been found that the torso base portion can have a lateral dimension of from 6 to 10 inches, in other embodiments, from 7 to 9 inches, and, in other embodiments, about 8 inches. In the range of from 6 to 10 inches, most individuals can be appropriately fitted with the torso base portion 34 anchored between scapulae.

The height (h, FIG. 4) of the torso base portion 34 and supine-suppression fin 36 is sized large enough to prevent the sleep fin 14 from flipping upwardly or downwardly against the wearer's back such that the interior base surface 38 would no longer be in contact with the wearer's back. In some embodiments, the height of the torso base portion 34 and supine-suppression fin 36 is greater than 3 inches, in other embodiments, greater than 4, and, in other embodiments, greater than 5. The height could be made to be the same as the height of an intended wearer's torso, though that would increase significantly the material costs of the device 10.

The depth (d, FIG. 4) of the sleep fin 14, from the interior base surface 38 to the apex 56 (i.e., not taking into account the spine indentation), is sized so that, when the sleep fin 14 is worn by an individual and that individual lies down so as to be supported by the apex 56 and either his right or left side (see FIG. 8), the plane of the interior base surface 38 extends at an angle of greater than 5 degrees relative to the support surface. In other embodiments, the angle is preferably greater than 10 degrees, in other embodiments greater than 15 degrees, and, in other embodiments, greater than 30 degrees, and, in other embodiments, greater than 45 degrees. In other embodiments, the angle is preferably between 15 and 80 degrees, in other embodiments, between 20 and 70 degrees, in other embodiments, from 40 to 60 degrees, and, in other embodiments, from 40 to 50 degrees.

In other embodiments, the depth of the sleep fin 14 is sized so that, when the sleep fin 14 is set against a flat surface and supported by the apex 56 and either the right or left distal end of the torso base portion 34 (See FIG. 8), the plane of the interior base surface 38 extends at an angle of greater than 5 degrees relative to the support surface. In other embodiments, the angle is preferably greater than 10 degrees, in other embodiments greater than 15 degrees, in other embodiments, greater than 30 degrees, and, in other embodiments, greater than 45 degrees. In order to ensure that one wearing the device 10 is not extremely frustrated in rolling over during sleep, the depth is preferably kept to 8 inches or less, in other embodiments, at 7 inches or less, in other embodiments, at 6 inches or less, and, in other embodiments, at 5 inches or less.

The sleep fin 14 may also be dimensioned in accordance with the bodily dimensions of the individual that is to wear the sleep fin. Thus, in some embodiments, the width w is from 30 to 100% of the width of the torso (at the scapular region) of the user, in other embodiments, from 30 to 70% of the width of the user's torso, in other embodiments, from 40 to 60% of the width of the user's torso.

In particular embodiments, the width w is from 6 to 18 inches, the height h is from 3 to 12 inches, the depth is from 2 to 8 inches, and the spine indentation is from ¼ to 1 inch in depth, and from 1 to 5 inches in width. In yet other embodiments, the width w is from 7 to 12 inches, the height h is from 4 to 8 inches, the depth is from 3 to 6 inches, and the spine indentation is from ½ to ¾ inch in depth, and from 2 to 4 inches in width. In a specific embodiment, the width w is 8 inches, the height h is 5 inches, the depth d is 3 inches, and the spine indentation is ¾ of an inch in depth and 3 inches in width.

In this particular embodiment the sleep fin 14 is a unitary element, the torso base portion 34 and the supine-suppression fin 36 being integral with each other. It should be appreciated that the sleep fin 14 could be made of a separate and distinct torso base portion 34 and a separate and distinct supine-suppression fin 36, with those portions being appropriately joined together to make a structurally sound sleep fin 14. In the present embodiment, the sleep fin 14 is formed of a single material, such that there is no significant bright-line distinction between where the torso base portion 34 and the supine-suppression fin 36 begins. Nevertheless, for purposes of disclosing and claiming this invention, it is helpful to have the torso base portion 34 defined as an element independent of the supine-suppression fin 36.

In some embodiments, the supine-suppression fin 36 is made of a stiff, non-deformable material. In other embodiments, the supine-supression fin 36 is somewhat resilient, though still of sufficient stiffness to retain its general parabolic or left-side and right-side concave shape. High density foam has been found to be an appropriate material, and, in particular embodiments, the foam has a force deflection of from 60 to 300 pounds (lbs) as measured by ASTM D3574. In accordance with other embodiments, the foam has a force deflection of from 60 to 250 lbs (ASTM D3574). In accordance with other embodiments, the foam has a force deflection of from 60 to 200 lbs (ASTM D3574). In accordance with other embodiments, the foam has a force deflection of from 60 to 150 lbs (ASTM D3574). In accordance with other embodiments, the foam has a force deflection of from 60 to 100 lbs (ASTM D3574), and in yet other embodiments, from 80 to 100 lbs (ASTM D3574). In other embodiments, the supine-supression fin 36 may be made of substantially rigid materials such that it does not deflect under force.

In alternative embodiments, the sleep fin 14 is formed of an inflatable bladder that, when pressurized with air, provides the general shape shown and disclosed herein. In such embodiments, the bladder of the sleep fin 14 would be inflated to a pressure to compress less than 10% of its depth d under the load of the weight of the user.

The sleep fin 14 must be stiff enough to significantly retain its shape when the wearer attempts to lie on top of the sleep fin 14 in a supine position. If the sleep fin 14 deforms too much (typically around 10 to 15% of its depth d) when the wearer attempts to lie in the supine position, it might be possible for the wearer to remain in the supine position. However, if the sleep fin 14 significantly retains its shape the wearer is highly unlikely to remain in a supine position, because the apex 56 provides very little surface area for support in a direct supine position. Instead, one wearing the therapeutic sleep device 10 is urged to lie either directly on their stomach or side, wherein the sleep fin 14 does not even contact the sleeping surface or mattress, or, as seen in FIG. 8, is urged to sleep in a position significantly angled off of the supine position, where the apex 56 and one of the left or right torso wings 44, 46 provide some support to the wearer.

Unlike many of the therapeutic sleep devices of the prior art, the therapeutic sleep device 10 does not significantly frustrate the ability of the wearer to move from a left-side sleeping position to a right-side sleeping position or vise versa, whether by rolling over their back or rolling over their chest. Because the supine-suppression fin 36 is shaped with convex surfaces joining at an apex aligned with the wearer's spine, the individual is able to move between various sleeping positions, but is always frustrated from stopping in a supine position, being urged instead onto a side or the stomach or to a position such as that shown in FIG. 8.

It will be recalled that, in the prior art devices such as those mentioned hereinabove, when rolling toward the supine position, the shape of the device is such that the pad abruptly engages the support surface (e.g. mattress), with a significant area of contact between the pad and support surface, and the individual thus experiences a transition from no resistance of movement (when the pad does not touch the support surface) to full resistance of movement (when the pad and support surface come into contact). This resistance will at least significantly frustrate movement toward or through the supine position, thus frustrating an individual's natural tendencies to roll over during sleep. In contrast, with the present invention, the shape of the sleep fin 14 is designed such that it only gradually engages the sleep support surface as the person attempts to roll into and through a supine position. Due to the narrow apex 56, the supine sleep position is unstable and a person using the device will continue to roll into a position sufficiently angled from the supine position to be effective. As seen in FIG. 8, as one wearing the sleep device 10 rolls over, the sleep fin 14 initially contacts the support surface M (e.g. mattress) at the distal end of one of the wings 44, 46 (46 in FIG. 8), and soon after, the apex 56 contact the support surface as well, providing those two points of contacts, as opposed to an intimate contact between large, generally flat surface areas. Because of the general parabolic-like shape of the sleep fin 14, it is easier to pivot about the apex 56 than to pivot about those structures of the prior art. Additionally, because the apex is aligned with the spine, it is difficult to stop in the supine position because the apex provides little surface area for support. As the weight of the wearer is pushing down, the wearer will tend to tilt to one side or the other, depending upon which side the weight is greatest. Notably, the soft cushion of the backing material ensures the comfortable use of the device as a person rolls through a supine position because of its ergonomic design, which reduces back pressure.

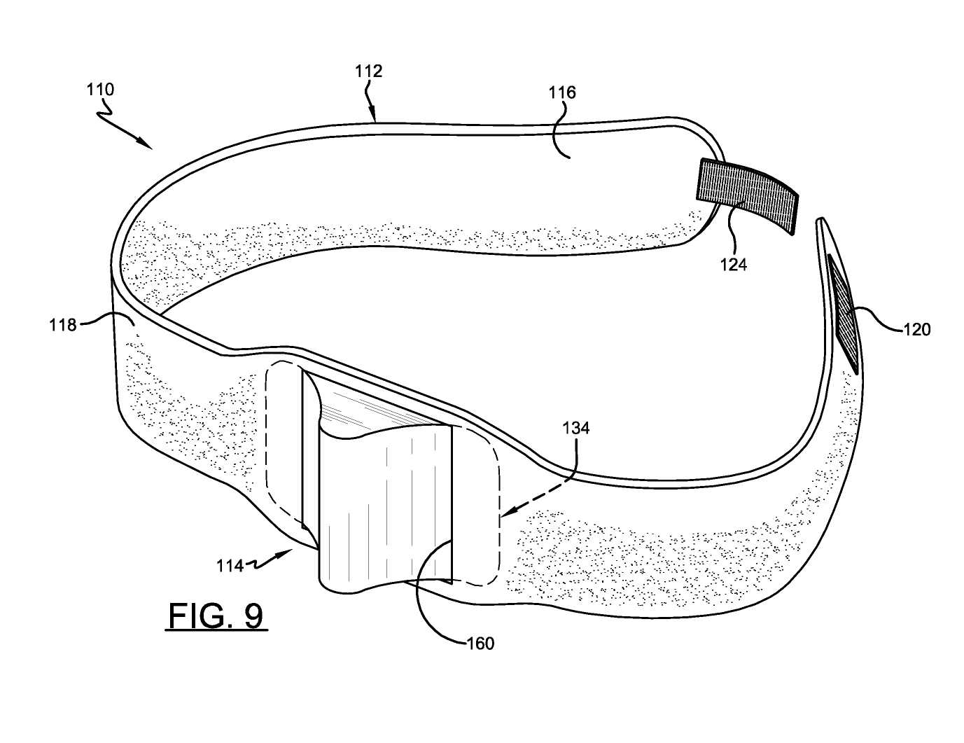

More particularly, the therapeutic sleep device 10 includes an interior back-contacting region that is of a soft material for cushioning the contact between the device 10 and the wearer's back. In this embodiment, the interior back-contacting region is provided by the body securing member 12, particularly at the back pad portion 28, but the interior back contacting region of soft material can be otherwise provided, as, for example, in the embodiment of FIGS. 9-11.

In many respects, the embodiment of FIGS. 9-11, is similar to the therapeutic sleep device 10 previously disclosed, but this embodiment differs in some respects as well. Due to the similarities between the embodiments, like parts in the second embodiment will receive like numerals though increased by 100. Thus, the therapeutic sleep device 110 includes a body securing member 112 and a sleep fin 114. The body securing member 12 provides an interior side 116 and a posterior side 118. Hook members 120 are provided at the posterior side 118, and hook members 124 are provided at the interior side 116 of the body securing member 12, and the body securing member can therefore be secured around the wearer's torso substantially as disclosed with respect to the embodiment of FIG. 1.

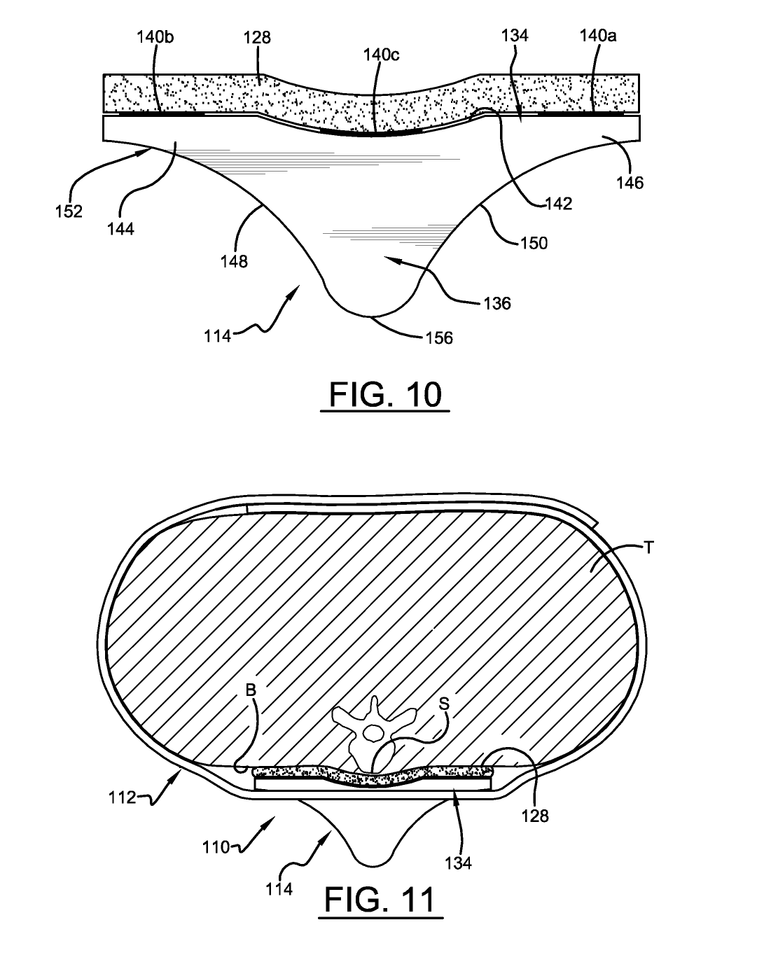

With reference to FIG. 10, the sleep fin 114 includes a torso base portion 134 and a supine-supression fin 136. The supine-supression fin 136 is shaped to define a spine indentation 142 that is centered between a left interscapula wing 144 and a right interscapula wing 146. The supine-suppression fin 136 provides a left convex surface 148 and a right convex surface 150, both of which taper from a wider base region 152 to a narrower peak region 154. At this peak region, the left and right convex surfaces 148, 150 join at an apex 156. The apex 156 is aligned (in the interior to posterior direction) with the spine indentation 142. As seen in FIG. 10, the sleep fin 114 receives a separate back pad portion 128 that, in this embodiment, is not provided as part of a body securing member 112. The back pad portion 128 is joined to the supine-suppression fin 136 by hook and loop fastening, as at hook members 140a, 140b, 140c, with the back pad portion 128 providing appropriate loop patches or being made covered with a fabric that provides a loop fastening. The back pad portion 128 is made of soft, cushioning materials 162, such as those disclosed above with respect to the back pad portion 28. This is particularly beneficial in this embodiment because, as will be discussed more fully below, the back pad portion 128 provides the surface that is placed directly against the wearer's back. The supine-suppression fin 136 is preferably made of materials as already disclosed with respect to fin 36.

In this embodiment, the sleep fin 114 is secured in place at the wearer's back by being pressed against the wearer's back by the body securing member 112. As seen in FIG. 9, the body securing member 112 includes a fin aperture 160 that is sized to permit the passage of the supine-suppression fin 136 and to resist the passage of the left and right torso wings 144, 146. Thus, as seen in FIG. 11, the sleep fin 114 can be positioned with the spine indentation 142 aligned with the wearer's spine S, and the body securing member 112 can be positioned over the sleep fin 114, with the supine-suppression fin 136 extending through the fin aperture 160 to be present posteriorly of the body securing member 112. In this way, the sleep fin 114, more particularly the back pad portion 128 is sandwiched between the wearer's torso T and the body securing member 112, such that significant pressure can be applied to bring the back pad portion 128 snugly against the wearer's back. Thus, like device 10, this therapeutic sleep device 110 also includes an interior back-contacting region that is of a soft material for cushioning the contact between the device 110 and the wearer's back. In this embodiment, the interior back-contacting region is provided by a separate back pad portion 128 secured to the torso base portion 134.

It should be appreciated that the back pad portion 128 could also be more permanently formed into the sleep fin 114 so as to be unitary therewith, with the back pad portion providing a soft material for engaging the wearer's back, and the remainder being formed of stiffer materials, as already disclosed. It is merely for ease of manufacturing that the back pad portion 128 is separate and distinct and secured to the sleep fin by hook and loop fastening.

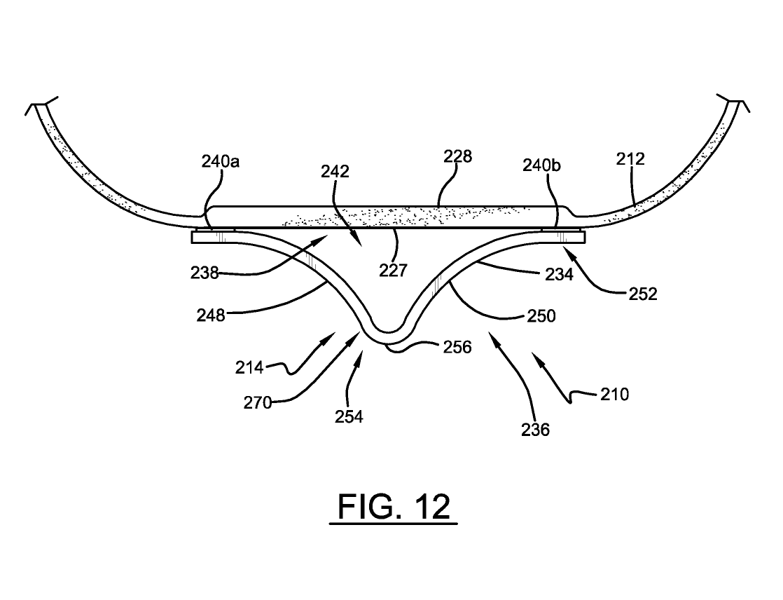

With reference to FIG. 12, a third embodiment of a therapeutic sleep device is shown and designated by the numeral 210. The therapeutic sleep device 210 includes a body securing member 212 and a sleep fin 214. The body securing member 212 can be broadly selected, as with body securing member 12 of the first embodiment, though it is shown here as a belt that would be substantially similar to the belt shown for body securing member 12. Sizing and the like would be substantially similar as well.

The sleep fin 214 is secured to the body securing member 212 at a back pad portion 228 thereof. In this embodiment, the torso base portion 234 and supine-suppression fin 236 are formed of a rigid curved member 270 defining an open interior base 238, which herein is to be understood as defining a spine indentation 242 The torso base portion 234 includes multiple patches of hook members 240a and 240b, much like sleep fin 14, though a central patch of hook members (like hook members 40c) is not available, in light of the open interior base 238. The hook members 240a and 240b can secure the sleep fin 214 to the back pad portion 228 because the body securing member 212 of this embodiment has an exterior surface made of a loop fabric 227. The supine-suppression fin 236 provides a left convex surface 248 and a right convex surface 250, both of which taper from a wider base region 252 to a narrower peak region 254. At this peak region, the left and right convex surfaces 248, 250 join at an apex 256, which is preferably slightly rounded, as shown, though it could be a sharper point, if desired. The apex 256 is aligned with the spine indentation 242. The rigid curved member 270 will be rigid and strong enough to maintain the curved shape to prevent a wearer from remaining in a supine position, as provided above with respect to other embodiments. Sizing and the like would be substantially similar to that of sleep fin 214. In this embodiment, the large spine indentation provides significant room to permit the body securing member to conform naturally to the backpad, because the backpad is free to move in the space provided by the spine indentation. This provides comfort and stability, protects the spine, and helps keep the sleep fin centered for proper functioning.

In light of the foregoing, it should be appreciated that the present invention significantly advances the art by providing a therapeutic sleep device that is structurally and functionally improved in a number of ways. While particular embodiments of the invention have been disclosed in detail herein, it should be appreciated that the invention is not limited to or by any such embodiments inasmuch as variations on the invention herein will be readily appreciated by those of ordinary skill in the art. The scope of the invention shall be appreciated from the claims that follow.

What is claimed is:

1. A therapeutic sleep device worn about an individual's torso and intended to discourage an individual from sleeping in a supine position, the therapeutic sleep device comprising:
a body securing member having a interior side and a posterior side; and
a sleep fin including a supine-suppression fin providing a right convex surface and a left convex surface tapering from a wider base region to a narrower peak region at which said right and left convex surfaces join at an apex, wherein said apex and at least a portion of said right and left convex surfaces extend beyond said posterior side of said body securing member.

2. The therapeutic sleep device of claim 1, wherein said sleep fin further includes a torso base portion defining a spine indentation centered between a right torso wing and a left torso wing, said spine indentation being aligned with said apex.

3. The therapeutic sleep device of claim 2, further comprising an interior back-contacting region of a soft material for cushioning the contact between the device and a wearer's back.

4. The therapeutic sleep device of claim 3, wherein said interior back-contacting region is provided by a back pad portion provided at said interior side of said body securing member.

5. The therapeutic sleep device of claim 4, wherein said back pad portion is integral with said body securing member.

6. The therapeutic sleep device of claim 4, wherein said back pad portion is secured to said torso base portion, the securing thereof being either removable or permanent.

7. The therapeutic device of claim 6, wherein said body securing member includes an aperture permitting the passage of said apex and at least a portion of said right and left convex surfaces to extend beyond said posterior side of said body securing member, and resisting the passage of said torso base portion.

8. A therapeutic sleep device worn about an individual's torso and intended to discourage an individual from sleeping in a supine position, the therapeutic sleep device comprising:
a body securing member having a interior side and a posterior side; and
a sleep fin including an apex and a torso base portion defining a spine indentation centered between a right torso wing and a left torso wing, said spine indentation being aligned with said apex.

9. The therapeutic sleep device of claim 8, said sleep fin further including a supine-suppression fin providing a right convex surface and a left convex surface tapering from a wider base region to a narrower peak region at which said right and left convex surfaces join at said apex, wherein said apex and at least a portion of said right and left convex surfaces extend beyond said posterior side of said body securing member

10. The therapeutic sleep device of claim 9, further comprising an interior back-contacting region of a soft material for cushioning the contact between the device and a wearer's back.

11. A therapeutic sleep device worn about an individual's torso and intended to discourage an individual from sleeping in a supine position, the therapeutic sleep device comprising:
a body securing member having a interior side and a posterior side; and
a sleep fin including:
a supine-suppression fin providing a right convex surface and a left convex surface tapering from a wider base region to a narrower peak region at which said right and left convex surfaces join at an apex, and
a torso base portion defining a spine indentation centered between a right torso wing and a left torso wing, said spine indentation being aligned with said apex,
wherein said apex and at least a portion of said right and left convex surfaces extend beyond said posterior side of said body securing member.

12. The therapeutic sleep device of claim 11, further comprising an interior back-contacting region of a soft material for cushioning the contact between the device and a wearer's back

13. The therapeutic sleep device of claim 12, wherein said interior back-contacting region is provided by a back pad portion provided at said interior side of said body securing member.

14. The therapeutic sleep device of claim 13, wherein said back pad portion is integral with said body securing member.

15. The therapeutic sleep device of claim 13, wherein said back pad portion is secured to said torso base portion, the securing thereof being either removable or permanent.

16. The therapeutic device of claim 15, wherein said body securing member includes an aperture permitting the passage of said apex and at least a portion of said right and left convex surfaces to extend beyond said posterior side of said body securing member, and resisting the passage of said torso base portion.
